(19) United States
(12) Patent Application Publication
(10) Pub. No.: US 2024/0252761 A1
(43) Pub. Date: Aug. 1, 2024

(54) DRUG DELIVERY DEVICE, PLUNGER ROD, SET OF PLUNGER RODS, METHOD FOR ASSEMBLING A DRUG DELIVERY DEVICE AND SET OF DRUG DELIVERY DEVICES

(71) Applicant: Sanofi, PARIS (FR)

(72) Inventors: Uwe Dasbach, Frankfurt am Main (DE); Timothy Denyer, Melbourn Hertfordshire (GB); Robin EINWÄCHTER, Frankfurt am Main (GB); Thomas Mark Kemp, Melbourn Hertfordshire (GB); Tom Lever, Melbourn Hertfordshire (GB); Robbie Wilson, Melbourn Hertfordshire (GB); Alex Zuyev, Bridgewater, NJ (US)

(21) Appl. No.: 18/565,747

(22) PCT Filed: May 31, 2022
(86) PCT No.: PCT/EP22/64805

§ 371 Date: Nov. 30, 2023

(30) Foreign Application Priority Data
Jun. 2, 2021 (EP) 21315094.9

(51) Int. Cl. A61M 5/315; A61M 5/20; A61M 5/32

(52) U.S. Cl. CPC A61M 5/3157; A61M 5/2033; A61M 5/31501; A61M 5/31515; A61M 5/31578; A61M 5/3245; A61M 5/3257; A61M 2005/2013; A61M 2005/206; A61M 2005/3247; A61M 2205/581; A61M 2205/582; A61M 2205/583; A61M 2207/10

(57) ABSTRACT

The invention relates to a drug delivery device (1000) comprising a housing (100) configured to receive a medicament container (8) with a stopper (82) proximally sealing the medicament container. Furthermore, the drug delivery device comprises a plunger rod (1) arranged axially movable with respect to the housing and/or the medicament container and a feedback mechanism operatively coupled to the plunger rod. The drug delivery device has an initial state in which the plunger rod is in a start position. The drug delivery device is configured such that, in the initial state and when the medicament container is received in the housing, the plunger rod is axially spaced from the stopper. Furthermore, the drug delivery device is configured such that the plunger rod is axially movable from its start position in a distal direction into a feedback position. The movement of the plunger rod in the distal direction enables the plunger rod to interact with the stopper in order to push the stopper in the distal direction such that a medicament stored in the medicament container is delivered. The feedback mechanism is triggered when the plunger rod reaches the feedback position. Triggering of the feedback mechanism creates an audible and/or tactile feedback in order to indicate an end of the medicament delivery. The invention also relates to a plunger rod, a set of plunger rods, a set of drug delivery devices and a method for assembling a drug delivery device.

### TECHNICAL FIELD

A drug delivery device is provided. Furthermore, a plunger rod, a set of plunger rods, a method for assembling a drug delivery device and a set of drug delivery devices is provided.

### BACKGROUND

Administering an injection is a process which presents a number of risks and challenges for users and healthcare professionals, both mental and physical. A drug delivery device may aim to make self-injection easier for patients. A conventional drug delivery device may provide the force for administering the injection by a spring and trigger button or another mechanism may be used to activate the injection. Drug delivery devices may be single-use or reusable devices.

There remains a need for an improved drug delivery device. Furthermore, there remains a need for a method for assembling such a drug delivery device, for an improved plunger rod and a set of plunger rods for such a drug delivery device as well as for a set of such drug delivery devices.

### SUMMARY

One object to be achieved is to provide an improved drug delivery device. Further objects to be achieved are to provide a plunger rod for such a drug delivery device, an improved set of plunger rods for drug delivery devices, an improved set of drug delivery devices and an improved method for assembling a drug delivery device. These objects are achieved, inter alia, by the subject matter of claims 1, 13, 14 and 15. Advantageous embodiments and further developments are subject of the dependent claims and are also presented in the following description and in the figures.

Firstly, the drug delivery device is specified. The drug delivery device may be a single-use device and/or a disposable device.

According to at least one embodiment, the drug delivery device comprises a housing configured to receive and/or hold a medicament container with a stopper. The stopper may seal the medicament container proximally, i.e. in the proximal direction. The drug delivery device may be configured to receive the medicament container in the housing such that the medicament container is axially and/or rotationally fixed with respect to the housing. The drug delivery device may comprise a medicament container holder which is a separate component from the housing and which is configured to receive and/or hold the medicament container.

The housing may comprise or consist of plastic and/or may be formed in one piece. The housing may be hollow and/or elongated and/or hollow cylindrically-shaped. The housing may be a sleeve. A longitudinal axis of the drug delivery device may run through the center of the housing. The housing may be formed in one piece, i.e. be of unitary construction or integrally formed. The medicament container holder may be received in the housing. The medicament container holder may be axially and/or rotationally fixed with respect to the housing. The medicament container holder may be formed in one piece.

According to at least one embodiment, the drug delivery device comprises a plunger rod arranged axially movable with respect to the housing and/or the medicament container. The plunger rod may be axially movable in only one or two opposite axial directions. The plunger rod may be hollow or solid. The plunger rod may be cylindrically-shaped, e.g. hollow cylindrically-shaped. In case that the plunger rod is hollow, further elements or members, e.g. an energy member for driving the plunger rod, may be received in the plunger rod. The plunger rod may comprise or consist of plastic. For example, the plunger rod is formed in one piece.

Here and in the following, if not stated otherwise, a movement of a member or element or feature is to be understood as a movement with respect to the housing.

For example, the plunger rod is received in the housing. The plunger rod may be circumferentially surrounded, e.g. circumferentially completely surrounded, by the housing. The housing may project beyond the plunger rod in a distal and/or a proximal direction. The plunger rod may have a main extension direction parallel to the longitudinal axis of the drug delivery device. The longitudinal axis may run through the plunger rod, e.g. through a center thereof.

According to at least one embodiment, the drug delivery device comprises a feedback mechanism operatively coupled to the plunger rod. The feedback mechanism may, in particular, be an audible and/or tactile feedback mechanism configured to indicate an end of a medicament delivery process. The feedback mechanism may be operatively coupled to the plunger rod such that a movement, e.g. an axial movement, of the plunger rod triggers the feedback mechanism. For example, the plunger rod may be in direct contact to the feedback mechanism or an element/member/feature thereof, respectively.

According to at least one embodiment, the drug delivery device has an initial state in which the plunger rod is in a start position. The start position may be a most proximal position of the plunger rod. The initial state may be an as-delivered state of the drug delivery device, i.e. a state in which a user of the drug delivery device receives the drug delivery device. In other words, in the initial state, the drug delivery device is not activated.

According to at least one embodiment, the drug delivery device is configured such that, in the initial state and when the medicament container is received in the housing and/or held by the housing, the plunger rod is axially spaced from the stopper. Thus, there is a clearance between the plunger rod and the stopper in the initial state. The clearance may compensate manufacturing tolerances, assembling tolerances and/or movement of the stopper during transport. In other words, there is a clearance between the plunger rod and the stopper in all tolerance conditions. Particularly, the plunger rod may then be arranged proximally offset with respect to the stopper. For example, a distal end of the plunger rod is spaced from a proximal end of the stopper in the initial state. In the initial state and when the medicament container is received in the housing, the stopper may also be in a respective start position, which may be a most proximal position of the stopper. In the initial state, the distal end of the plunger rod may be arranged within the medicament container or outside of the medicament container.

According to at least one embodiment, the drug delivery device, particularly when starting from the initial state, is configured such that the plunger rod is axially movable from its start position in the distal direction into a feedback position. The feedback position may be an end position of the plunger rod or a position between the end position and the start position. The feedback position and/or the end position may be located distally with respect to the start position. The end position may be the most distal position of the plunger rod when the drug delivery device is used. If the feedback position lies between the end position and the start position, the feedback position is preferably located closer to the end position than to the start position.

According to at least one embodiment, the drug delivery device is configured such that the movement of the plunger rod in the distal direction enables the plunger rod to interact with the stopper. The interaction may be thus that the plunger rod pushes the stopper, e.g. from its start position, in the distal direction when the plunger rod is moved in the distal direction. Preferably, the movement of the stopper in the distal direction results in a delivery of a medicament stored in the medicament container.

In other words, when the medicament container is received in the housing, a distal movement of the plunger rod may lead to an interaction between the plunger rod and the stopper so that the movement or a further movement of the plunger rod in the distal direction pushes the stopper in the distal direction in order to deliver a drug stored in the medicament container.

For example, the interaction between the plunger rod and the stopper may be an impact or an abutment between the plunger rod and the stopper. For example, when the plunger rod interacts with the stopper, the distal end of the plunger rod directly contacts a proximal end of the stopper. The drug delivery device may be configured such that the interaction happens before the plunger rod reaches the feedback position.

According to at least one embodiment, the drug delivery device is configured such that the feedback mechanism is triggered or activated, respectively, when the plunger rod reaches the feedback position. The feedback mechanism may be triggered due to the operative coupling between the plunger rod and the feedback mechanism. Preferably, the feedback mechanism is only triggered or can only be triggered when the plunger rod is in the feedback position. The triggering of the feedback mechanism may happen automatically due to the plunger rod reaching the feedback position.

According to at least one embodiment, triggering of the feedback mechanism creates an audible and/or tactile feedback in order to indicate an end of the medicament delivery. The audible and/or tactile feedback may, in particular, be such that it is noticeable for a user of the drug delivery device. The feedback may provide an unambiguous indication of the end of the medicament delivery. For example, the audible feedback is at least 70 dB or at least 80 dB or at least 90 dB.

In at least one embodiment, the drug delivery device comprises a housing configured to receive a medicament container with a stopper proximally sealing the medicament container. Furthermore, the drug delivery device comprises a plunger rod arranged axially movable with respect to the housing and/or the medicament container and a feedback mechanism operatively coupled to the plunger rod. The drug delivery device has an initial state in which the plunger rod is in a start position. The drug delivery device is configured such that, in the initial state and when the medicament container is received in the housing, the plunger rod is axially spaced from the stopper. Furthermore, the drug delivery device is configured such that the plunger rod is axially movable from its start position in a distal direction into a feedback position. The movement of the plunger rod in the distal direction enables the plunger rod to interact with the stopper in order to push the stopper in the distal direction such that a medicament stored in the medicament container is delivered. The feedback mechanism is triggered when the plunger rod reaches the feedback position. Triggering of the feedback mechanism creates an audible and/or tactile feedback in order to indicate an end of the medicament delivery.

The present invention is, inter alia, based on the recognition that, when the device is designed such that the plunger rod is intentionally axially spaced from the stopper in an initial state, manufacturing tolerances, e.g. in the position of the stopper due to variations in the fill volume of the drug and/or variations in the length of the plunger rod, do not negatively affect the usability of the drug delivery device. Also displacements of the stopper during transport, e.g. due to a change of the drug volume and/or the volume of gas bubble in the cartridge because of changes in the ambient pressure, can be absorbed by such a spacing. Indeed, a contact of the plunger rod and the stopper before an intended use of the drug delivery could lead to leak during storage or to drug emitted prior to the intended usage.

The drug delivery device specified herein may be elongated and/or may comprise a longitudinal axis, i.e. a main extension axis. An axial direction may be a direction parallel to the longitudinal axis. By way of example, the drug delivery device may be cylindrically-shaped.

Furthermore, the drug delivery device may comprise an end, e.g. a longitudinal end, which may be provide to face or to be pressed against a skin region of a human body. This end is herein called the distal end. A drug or medicament may be supplied via the distal end. The opposing end is herein called the proximal end. The proximal end is, during usage, remote from the skin region. The axial direction pointing from the proximal end to the distal end is herein called distal direction. The axial direction pointing from the distal end to the proximal end is herein called proximal direction. A distal end of a member or element or feature of the drug delivery device is herein understood to be the end of the member/element/feature located most distally. Accordingly, the proximal end of a member or element or feature is herein understood to be the end of the element/member/feature located most proximally.

In other words, “distal” is used herein to specify directions, ends or surfaces which are arranged or are to be arranged to face or point towards a dispensing end of the drug delivery device or components thereof and/or point away from, are to be arranged to face away from or face away from the proximal end. On the other hand, “proximal” is herein used to specify directions, ends or surfaces which are arranged or are to be arranged to face away from or point away from the dispensing end and/or from the distal end of the drug delivery device or components thereof. The distal end may be the end closest to the dispensing end and/or furthest away from the proximal end and the proximal end may be the end furthest away from the dispensing end. A proximal surface may face away from the distal end and/or towards the proximal end. A distal surface may face towards the distal end and/or away from the proximal end. The dispensing end may be a needle end where a needle unit is or is to be mounted to the device, for example.

A direction perpendicular to the longitudinal axis and/or intersecting with the longitudinal axis is herein called radial direction. An inward radial direction is a radial direction pointing towards the longitudinal axis. An outward radial direction is a radial direction pointing away from the longitudinal axis.

The terms “angular direction”, “azimuthal direction” or “rotational direction” are herein used as synonyms. Such a direction is a direction perpendicular to the longitudinal axis and perpendicular to the radial direction.

An element or member or feature being rotationally, axially or radially fixed with respect to another element or member or feature means that a relative movement in rotational direction or axial direction or radial direction between the two elements/members/features is not possible or prevented.

The terms “protrusion” and “boss” are used as synonyms herein. The term “recess” may particularly stand for an indentation or a cut-out or opening or hole.

According to at least one embodiment, the drug delivery device is configured such that, when the medicament container is received in the housing, the plunger rod is movable from its start position in the distal direction by a travel distance before interacting with the stopper. The drug delivery device may be configured like this at least when starting from the initial state.

For example, the plunger rod may be movable from its start position in the distal direction by the travel distance until reaching an interaction position. For example, during movement of the plunger rod from the start position to the interaction position, the stopper is not moved in distal direction. Only when reaching the interaction position, the interaction between the plunger rod and the stopper may start. From there on, further movement of the plunger rod in the distal direction may push the stopper in the distal direction. When reaching the interaction position, the plunger rod may hit against the stopper and may abut against the stopper. In particular, the interaction position is axially located between the feedback position and the start position, e.g. closer to the start position than to the feedback position.

According to at least one embodiment, in the initial state and when the medicament container is received and/or held in the housing, the space between the plunger rod and the stopper is gas-filled. Preferably, however, the space is not sealed but vented to atmosphere.

According to at least one embodiment, after having been moved by the travel distance and before reaching the feedback position, the plunger rod interacts with the stopper, in particular pushes the stopper in the distal direction.

According to at least one embodiment, the drug delivery device is configured such that, in the initial state and when the medicament container is received in the housing, an initial distance between the plunger rod and the stopper is chosen such that the probability of a damage of the medicament container upon interaction of the plunger rod with the stopper is reduced. For example, a damage is prevented. The initial distance may be equal to the travel distance.

When the plunger rod is moved from its start position in distal direction, the plunger rod may hit the stopper with a certain force. An impulse is transferred to the medicament container. As the medicament container may comprise glass, e.g. may comprise a cartridge formed of glass, the impulse could lead to a breakage of the glass. In the present invention, however, the initial distance between the plunger rod and the stopper is preferably chosen such, e.g. such small, that the risk of a damage, e.g. a breakage, of the medicament container is reduced. However, the initial distance is chosen sufficiently large to allow for the compensation of manufacturing and transport tolerances as mentioned above.

For example, the initial distance between the plunger rod and the stopper in the initial state is chosen such that a damage of the medicament container upon interaction of the plunger rod with the stopper is prevented.

According to at least one embodiment, in the initial state, an initial distance between the plunger rod and the stopper is chosen such that variations in the distance due to manufacturing tolerances and/or assembling tolerances and/or fluctuations occurring during a transport of the drug delivery device are taken into account. This means that the space between the stopper and the plunger rod is predetermined to absorb such variations. For example, the initial distance of the stopper and the plunger rod is chosen such that, if one or more or all of these variations occur and/or sum up, still a minimum distance between the plunger rod and the stopper is maintained, i.e. a direct contact between the plunger rod and the stopper is prevented. For example, a contact between the plunger rod and the stopper only appears upon activation of the drug delivery device, when switching from the initial state into a released state.

The distance between the plunger rod and the stopper may be subject to certain variations. One reason for these variations can be manufacturing tolerances of the drug delivery device, particularly of components thereof, like the length of the plunger rod or the fill volume of the medicament container. The fill volume influences the position of the stopper in the initial state. Also during assembling of the drug delivery device, variations may occur, e.g. du to varying compression of a drive spring of the drug delivery device. Furthermore, during transport of the device, the position of the stopper may change due to a change in the drug volume or a volume of a gas bubble in the cartridge, e.g. induced by varying ambient pressure.

According to at least one embodiment, the feedback mechanism comprises an indicator having a first state and a second state. The first state may be a biased state of the indicator, the second state may be a relaxed state of the indicator.

According to at least one embodiment, the drug delivery device is configured such that, when the feedback mechanism is triggered or activated, the indicator switches from the first state into the second state. This switching may create the audible and/or tactile feedback. For example, in the first state, the indicator has stored energy which is released in the form of an audible and/or tactical feedback when switching into the second state. For example, in the first state, the indicator is tensioned and stores tension energy. In the first state, the indicator may be deformed with respect to the second state.

It is also possible, that, when the indicator switches from the first state into the second state, the indicator interacts with, e.g. hits against, a further feature or element or member of the drug delivery device which creates the audible and or tactile feedback.

According to at least one embodiment, in the initial state, the indicator is in the first state. Alternatively, the indicator may be brought in the first state, e.g. from the second state, when the plunger rod is moved from its start position in the distal direction and may then switch back into the first state when the plunger rod reaches the feedback position.

According to at least one embodiment, the indicator comprises or is a resilient element or a resilient member. For example, the indicator comprises a spring or is a spring, e.g. a leaf spring. The indicator may comprise or consist of metal or plastic, e.g. may be a metal sheet. The indicator may be a monostable or a bistable spring element.

According to at least one embodiment, the indicator is bent by a certain angle about a longitudinal axis forming a longitudinal round fold and has two adjacent angled wing-shaped sections arranged on both sides of the longitudinal round fold. The indicator may furthermore comprise a notch in the longitudinal round fold. The notch may extend transversely to the longitudinal round fold. The notch may be arranged centrically in the longitudinal round fold. The indicator may comprise supporting tabs, e.g. at the wing-shaped sections. The supporting tabs may protrude outwardly from the wing-shaped section. The longitudinal round fold may have a bent radius between 0.5 mm and 5 mm, preferably between 1.5 mm in 2 mm, inclusive. The indicator may have a rectangular shape, a square shape or an oval shape.

According to at least one embodiment, the feedback mechanism comprises a trigger feature. The trigger feature may be part of the plunger rod, e.g. formed integrally with a main body of the plunger rod. The trigger feature may be axially and/or rotationally fixed with respect to the plunger rod.

According to at least one embodiment, the feedback mechanism comprises an operation feature. The operation feature may be axially and/or rotationally fixed with respect to the housing. The operation feature may be part of the indicator, e.g. integrally formed with the indicator, or may be part of a further element or member of the feedback mechanism.

According to at least one embodiment, at least one of the trigger feature and the operation feature is displaceable, i.e. is a displaceable feature. For example, the displaceable feature is displaceable in radial direction. The other feature may be radially fixed with respect to the plunger rod or the housing, respectively. The displaceable feature may be fixed to or may be part of a displaceable element, e.g. of a flexible or pivotable or resilient element, such as a flexible or pivotable or resilient arm. The displaceable element may be part of the plunger rod or may be axially and/or rotationally fixed with respect to the housing. For example, the displaceable element is axially orientated. The displaceable element may be elongated, e.g. with a main extension direction along the longitudinal axis. The displaceable feature may be located at the distal end of the displaceable element or may be located closer to the distal end than to the proximal end of the displaceable element. The distal end of the displaceable arm may be displaceable and the proximal end of the displaceable arm may radially fixed with respect to the housing.

According to at least one embodiment, the drug delivery device is configured such that, when the plunger rod is moved from its start position into the feedback position, the trigger feature and the operation feature axially pass each other or at least axially and/or rotationally overlap with each other which enables the displaceable feature to displace in order to trigger the feedback mechanism. The drug delivery device may be configured like this at least when starting from the initial state. The displacement of the displaceable feature may trigger or may enable a triggering of the feedback mechanism. The trigger feature and the operation feature may engage with each other when they axially and/or rotationally overlap with each other. For example, displacement of the displaceable feature enables or accompanies the transition of the indicator from the first state into the second state.

For example, when the plunger rod, starting from its start position, is moved in distal direction and reaches the feedback position, the trigger feature and the operation feature have passed each other or at least axially and/or rotationally overlap with each other. As a result, the displaceable feature displaces and the feedback mechanism is triggered.

According to at least one embodiment, the displaceable feature is held in a first position before the feedback mechanism is triggered, e.g. before the plunger rod reaches the feedback position. In the first position, the displaceable feature may be biased towards a second position, e.g. in radial inward or radial outward direction. The first position and the second position may be offset with respect to each other in radial direction.

According to at least one embodiment, the displaceable feature displaces from the first position into the second position when the plunger rod reaches the feedback position. The displacement may happen automatically, e.g. when the displaceable feature is biased in the first position towards the second position.

According to at least one embodiment, the operation feature is displaceable, e.g. in radial direction. The plunger rod may hold the displaceable operation feature in its first position when the plunger rod is at a position proximal with respect to the feedback position. For example, the displaceable operation feature may radially abut against a surface, e.g. an outer surface, of the plunger rod. This abutment may hold the displaceable operation feature in the first position. For example, the displaceable operation feature is held in the first position as long as the plunger rod is between the start position and the feedback position.

Alternatively, it is also possible that the trigger feature is displaceable, e.g. in a radial direction. The displaceable trigger feature may abut against a surface axially and/or rotationally fixed with respect to the housing when the plunger rod is in a position proximal with respect to the feedback position.

The displaceable feature may also be guided from the first position into the second position by a guiding feature, e.g. a ramp or rail, when the plunger rod reaches the feedback position. In this case, the displaceable feature, when it is in the first position, may or may not be biased towards the second position.

According to at least one embodiment, the operation feature is a protrusion, e.g. a radially projecting protrusion. For example, the protrusion projects from a displaceable element in radial inward or radial outward direction. The protrusion may have the shape of a fin. For example, the protrusion comprises a distal edge or surface facing in distal direction and a proximal edge or surface facing in proximal direction. The distal edge or surface may be steeper than the proximal edge or surface, e.g. when measured with respect to the longitudinal axis.

According to at least one embodiment, the trigger feature is a recess in the plunger rod. The recess may be elongated, e.g. with a main extension direction parallel to the main extension direction of the plunger rod and/or parallel to the longitudinal axis. The recess may be a groove or a cut-out. The recess may be in a lateral surface of the plunger rod. The recess may extend from the lateral surface towards or into a cavity in the plunger rod. The recess may be delimited in distal and/or proximal direction by edges of the plunger rod. It is also possible that the recess is only delimited in distal direction by such an edge and is open in proximal direction. In this case, the recess may extend to a proximal end of the plunger rod.

According to at least one embodiment, the trigger feature is a proximal end of the plunger rod, e.g. a proximally facing surface of the plunger rod.

It is also possible that the trigger feature is a protrusion as specified above and the operation feature is a recess as specified above.

According to at least one embodiment, when the plunger rod reaches the feedback position, the protrusion displaces into the recess or displaces into a space proximally behind the proximal end of the plunger rod. In particular, the recess and the protrusion are dimensioned to allow an engagement between the two features.

According to at least one embodiment, the feedback mechanism comprises a support element. The support element may provide a mechanical support for the indicator in order to hold the indicator in the first state before the plunger rod reaches the feedback position. For example, the support element and the indicator are in direct contact when the support element provides the mechanical support.

According to at least one embodiment, the mechanical support holding the indicator in the first state is released or resolved when the plunger rod reaches the feedback position so that the indicator is enabled to switch into the second state. In other words, when the plunger rod reaches the feedback position, the support element no longer holds the indicator in the first state so that the indicator switches or is enabled to switch into the second state. When the indicator is in the second state, the support element may again carry the indicator.

The support element may be axially and/or rotationally fixed with respect to the housing. The support element may be part of a member or element axially and/or rotationally fixed with respect to the housing, like an energy member holder or drive spring holder, respectively. The support element may be or may comprise the displaceable element mentioned above. The displaceable feature may be part of the displaceable element. In this sense, a displacement of the displaceable element may be correlated with a displacement of the displaceable feature and vice versa.

For example, the displaceable element is held in a first position when the plunger rod is in a position proximal with respect to the feedback position. The first position may be a biased position in which the displaceable element is biased towards a second position, e.g. in radial inward radial or radial outward direction. When the plunger rod reaches the feedback position, the displaceable element may be allowed to displace into the second position.

According to at least one embodiment, the operation feature is fixed to or is part of the support element.

According to at least one embodiment, the indicator is axially and/or rotationally fixed with respect to the housing.

According to at least one embodiment, the plunger rod is arranged rotatably with respect to the housing. The rotational axis of the plunger rod may define or may coincide with the longitudinal axis.

According to at least one embodiment, in the initial state, the plunger rod is coupled to the housing by an axial-lock interface. The axial-lock interface prevents an axial movement of the plunger rod in the distal direction. The axial-lock interface may be formed directly between the plunger rod and the housing or directly between the plunger rod and a member/element axially fixed to the housing, like a drive spring holder.

According to at least one embodiment, the drug delivery device is configured such that a rotation of the plunger rod relative to the housing releases the axial-lock interface and enables the movement of the plunger rod in the distal direction. For example, the plunger rod has to be rotated by at least 5° in order to release the axial-lock interface.

According to at least one embodiment, the plunger rod comprises a holding structure. The holding structure may be located in the region of the proximal end of the plunger rod, e.g. closer to the proximal end than to the distal end. The holding structure may comprise one or more holding features. The holding structure may comprise two or more holding features which are of offset with respect to each other in rotational and/or axial direction. For example, the holding structure comprises at least two or at least four or at least six holding features. Each holding feature may be a protrusion, e.g. a protrusion projecting from a main body of the plunger rod in radial outward direction. The protrusion(s) may project from a lateral surface of the plunger rod in radial outward direction.

According to at least one embodiment, the drug delivery device comprises an axial-lock feature axially and/or rotationally fixed with respect to the housing. The axial-lock feature may be part of the housing or of the drive spring holder. The axial-lock feature may be a recess.

According to at least one embodiment, in the initial state, the holding structure, particularly at least one holding feature thereof, and the axial-lock feature interact with each other or are engaged with each other such that the axial-lock interface is established. For example, a protrusion of the holding structure projects into the recess and abuts against an edge delimiting the recess in distal direction thereby preventing an axial movement of the plunger rod.

According to at least one embodiment, a rotation of the plunger rod resolves the interaction or the engagement between the holding structure and the axial-lock feature.

According to at least one embodiment, the drug delivery device comprises a medicament container. The medicament container may comprise a needle. The medicament container may be received in the housing, e.g. circumferentially surrounded by the housing. The needle may form a distal end of the medicament container. The medicament container may be arranged axially and/or rotationally fixed with respect to the housing, e.g. it is not moved with respect to the housing during the intended usage of the drug delivery device. Alternatively, the medicament container may be movable with respect to the housing and may be moved with respect to the housing during the intended usage of the drug delivery device.

The medicament container may be a syringe, e.g. a prefilled syringe. The medicament container may comprise glass. The medicament container may be proximally sealed by a stopper, i.e. an end of the medicament container opposite the needle is sealingly closed by the stopper. The medicament container may comprise a drug or medicament, e.g. a liquid drug or medicament. The stopper may be in contact with the medicament. The drug delivery device may be configured to empty the medicament container when a drug delivery process is performed. For example, the medicament container comprises a medicament in an amount sufficient for just one drug delivery operation.

According to at least one embodiment, the drug delivery device is an auto-injector.

According to at least one embodiment, the drug delivery device comprises an energy member configured to provide energy to induce an axial movement of the plunger rod in the distal direction. The energy member may be a drive spring, e.g. a compression spring, or another component configured to induce an axial movement of the plunger rod, e.g. a gas cartridge or an electric motor. The drive spring may be formed of metal, e.g. steel. The longitudinal axis may run through the center of the drive spring. The energy member may be received in the housing. The drive spring may be received in a cavity of the plunger rod.

According to at least one embodiment, the drug delivery device is configured to be switched from the initial state into a released state in which the plunger rod moves in distal direction due to the energy provided by the energy member. For example, in the initial state, the drive spring is biased, e.g. compressed, and may also bias the plunger rod in distal direction. In the released state, the biased drive spring may be released and may transfer energy to the plunger rod and move the plunger rod in distal direction. A release of the axial-lock interface may result in a switching from the initial state into the released state.

A distal end of the drive spring may abut against or may be secured to a proximally facing surface of the plunger rod. A proximal end of the drive spring may abut against or may be secured to the housing or the drive spring holder.

According to at least one embodiment, the drug delivery device comprises a release member, e.g. a needle shroud, arranged axially movable with respect to the housing. The release member may be received in the housing. The release member may be telescopically coupled to the housing. The release member may be rotationally fixed to the housing.

According to at least one embodiment, when the drug delivery device is in the initial state, movement of the release member in an axial direction, e.g. in the proximal direction, enables a rotation of the plunger rod in order to release the axial-lock interface. For example, in the initial state, the plunger rod is coupled to the housing by a rotation-lock interface which prevents a rotation of the plunger rod relative to the housing. The movement of the release member in an axial direction may release the rotation-lock interface in order to enable a rotation of the plunger rod.

For example, in the initial state, the plunger rod is biased in a rotational direction but prevented from its rotation by the rotation-lock interface. After release of the rotation lock-interface, the plunger rod may rotate automatically which, in turn, releases the axial-lock interface. The rotation-lock interface may be formed directly between the release member and the plunger rod. For example, the release member comprises a rotation-lock feature which, in the initial state, interacts or engages with the holding structure of the plunger rod, e.g. at least one holding feature thereof, thereby establishing the rotation-lock interface. Axial movement of the release member may resolve the interaction or the engagement.

The rotation-lock feature of the release member may be a recess in the release member into which a protrusion of the holding structure projects. The recess may be L-shaped. The protrusion may abut against an edge of the recess delimiting the recess in a rotational direction. This prevents a rotation of the plunger rod. When the release member is moved in axial direction, the edge of the recess may be displaced with respect to the protrusion of the holding structure so that a rotation of the plunger rod is enabled.

According to at least one embodiment, in the initial state, the release member is in an extended position in order to cover a drug delivery element of the medicament container. The drug delivery element may be a needle or cannula. For example, in the initial state, the release member distally projects beyond the drug delivery element when the medicament container is received in the housing. In the extended position, the release member may project distally beyond the housing. At least a distal region of the release member may be sleeve-shaped.

According to at least one embodiment, a movement of the release member in an axial direction, e.g. in the proximal direction, and into a retracted position enables the rotation of the plunger rod in order to release the axial-lock interface.

According to at least one embodiment, the retracted position of the release member is a position for exposing the drug delivery element. Thus, if the medicament container is received in the housing and the release member is in the retracted position, the drug delivery element is exposed. For example, the drug delivery element distally projects beyond the release member when the release member is in the retracted position.

According to at least one embodiment, the drug delivery device comprises a shroud spring. The shroud spring may be coupled to the release member and the housing. The shroud spring may be configured such that it induces a restoring force acting in an axial direction, e.g. in distal direction, on the release member when the release member is moved from the extended position towards the retracted position.

The drug delivery device may be used as follows: First, the drug delivery device is in its initial state. Then, a user presses the distal end of the drug delivery device against a skin region of a body, e.g. a human body. At this state, the distal end of the drug delivery device may be formed by a distal end of the release member. This forces the release member to move from the extended position into the retracted position. This movement biases the shroud spring and the biased shroud spring biases the release member in distal direction with respect to the housing. In the retracted position, the rotation-lock interface is released. As a consequence, the plunger rod rotates which, in turn, releases the axial-lock interface so that the drug delivery device switches into the released state. In the released state, the plunger rod may first move in distal direction without pushing the stopper in the distal direction. Then the plunger rod interacts with the stopper and pushes the stopper in distal direction such that the drug is delivered, e.g. injected into the tissue of the body. At or near the end position, the plunger rod reaches the feedback position which triggers the feedback mechanism and creates the audible and/or tactile feedback. This informs the user that the drug delivery process is finished. The user may now remove the distal end of the drug delivery device from the skin. The shroud spring forces the release member to move in distal direction, e.g. back into the extended position.

Next, the plunger rod for a drug delivery device is specified. The plunger rod may the plunger rod of the drug delivery device specified above. Therefore, all features disclosed in connection with the plunger rod of the drug delivery device specified above are also disclosed for the plunger rod specified in the following and vice versa.

According to at least one embodiment, the plunger rod comprises a trigger feature configured to enable a triggering of an audible and/or tactile feedback mechanism of the drug delivery device when the plunger rod is assembled into the drug delivery device and when the trigger feature axially passes or at least axially overlaps with an operation feature of the feedback mechanism.

According to at least one embodiment, the plunger rod comprises a holding structure configured to interact with an axial-lock feature of the drug delivery device in order to establish an axial-lock interface which prevents an axial movement of the plunger rod relative to the housing of the drug delivery device in at least one axial direction, e.g. in distal direction. The holding structure may also be configured to interact with a rotation-lock feature, e.g. of a release member, in order to establish a rotation-lock interface which prevents a rotation of the plunger rod relative to the housing and/or relative to the release member of the drug delivery device.

According to at least one embodiment, the trigger feature is a proximal end of the plunger rod and/or is not axially overlapping with the holding structure and/or proximally projects beyond the holding structure.

The region of the holding structure may be defined by the holding features of the holding structure. For example, a distal end of the holding structure is defined by the holding feature located most distally or by the distal end of this holding feature, respectively. A proximal end of the holding structure may be defined by the holding feature located most proximally or by the proximal end of this holding feature, respectively.

Next, a set of the plunger rods for drug delivery devices is specified. The set comprises a plurality of plunger rods, e.g. at least two or at least four or at least six plunger rods. All features disclosed in connection with the plunger rod of the above-described drug delivery device or with the above-described plunger rod are also disclosed for the plunger rods of the set of the plunger rods and vice versa. Particularly, the plunger rods of the set of plunger rods described in the following can be used for the above-described drug delivery device.

According to at least one embodiment, each plunger rod of the set of plunger rods comprises a trigger feature configured to enable a triggering of an audible and/or tactical feedback mechanism of the drug delivery device when the plunger rod is assembled in the drug delivery device and when the trigger feature axially passes or at least axially overlaps with an operation feature of the feedback mechanism.

According to at least one embodiment, each plunger rod of the set of plunger rods has a distal end. The distal end is, in particular, configured to interact with a stopper of a medicament container. The distal end is, in particular, configured to face a stopper of a medicament container. Preferably, each distal end of each plunger rod of the set of plunger rods is configured to interact with the stopper of the medicament container, when the drug delivery device is activated. Consequently, the drug delivery device and/or at least one plunger rod of the set of plunger rods is configured such that there is a clearance between the at least one plunger rod of the set of the plunger rods and the stopper in the initial state of the drug delivery device for every combination of plunger rod and medicament container.

According to at least one embodiment, at least some plunger rods of the set of plunger rods have different lengths. For example, the lengths vary by at least 10% or at least 20% or at least 50% around an average length averaged over all plunger rods of the set of plunger rods. For example, plunger rods of different lengths deviate in their lengths by at least 10% or at least 20% and/or by at least 5 mm or at least 10 mm.

According to at least one embodiment, a distance between the distal end and the trigger feature is the same or substantially the same for all plunger rods of the set of plunger rods. The distance is, e.g., measured from the distal end of the plunger rod to a distal end of the trigger feature.

Here and in the following, “substantially the same” means that deviations may occur, which are, e.g., at most 5% or at most 2% or at most 1% from an average value. The average value may be averaged over all members of the respective set. Furthermore, if two elements, members or features are claimed to be the same or substantially the same, this may also mean that the materials are the same.

Since the distance between the distal end and the trigger feature is the same or substantially the same for all plunger rods, most components of the drug delivery device, e.g. the housing, the operation feature, the release member and so on do not have to be exchanged when changing the length of the plunger rod. Changing the length of the plunger rod may be required when changing a fill volume of a medicament stored in the medicament container. Indeed, the feedback mechanism will trigger or activate regardless of the length of the plunger rod.

According to at least one embodiment, each of the plunger rods of the set of plunger rods comprises a holding structure. The holding structure may be the same or substantially the same for all plunger rods. For example, the positions of the holding features relative to each other in axial and/or rotational direction and/or the dimensions of the holding features are the same or substantially the same for all plunger rods.

Here and in the following, positions of the member, elements or features may be defined by the positions of their respective centers, e.g. centers of gravity.

Next, the method for assembling a drug delivery device is specified. Particularly, a drug delivery device as specified above may be assembled with the method described in the following. Therefore, all features disclosed in connection with the drug delivery device are also disclosed for the method and vice versa.

According to at least one embodiment, the method comprises a step in which a medicament container comprising a medicament is provided. The medicament occupies a fill volume in the medicament container. For example, the fill volume or the amount of the medicament, respectively, is between 0.5 mL and 2 mL, inclusive. The medicament container may comprise a stopper proximally sealing the medicament container. The stopper may be formed of a rubber and/or may be formed in one piece.

According to at least one embodiment, the method comprises a step in which a plunger rod from the set of plunger rods is chosen depending on the fill volume or the amount of the medicament, respectively. Particularly, the length of the plunger rod is chosen depending on the fill volume. For example, the smaller the fill volume the larger the length of the plunger rod is chosen.

According to at least one embodiment, the method comprises a step in which the drug delivery device is assembled by using the medicament container and the chosen plunger rod.

According to at least one embodiment, the provided medicament container is chosen from a set of medicament containers comprising a plurality of medicament containers. At least some of the medicament containers may comprise medicament occupying different fill volumes, i.e. may comprise different amounts of the medicament. The geometry or the dimensions of the different medicament containers may be the same or substantially the same for all medicament containers. Only the amount of the medicament and accordingly the position of the stopper may be different in different medicament containers.

Next, the set of drug delivery devices is specified. The set comprises a plurality of drug delivery devices, e.g. at least two or at least four or at least six. Each drug delivery device may be assembled with the method specified above. Therefore, all features disclosed in connection with the method are also disclosed for the drug delivery devices of the set of drug delivery devices and vice versa.

According to at least one embodiment, each drug delivery device of the set of drug delivery devices comprises a plunger rod. The plunger rod may be one of the above specified plunger rods, e.g. a plunger rod of the set of plunger rods.

According to at least one embodiment, each drug delivery device of the set of drug delivery devices comprises an audible and/or tactical feedback mechanism with an operation feature. Preferably, the operation feature is the same or substantially the same in all drug delivery devices. The feedback mechanism may, in each case, also comprise an indicator. Also the indicator may be the same or substantially the same in all drug delivery devices.

According to at least one embodiment, each drug delivery device of the set of the drug delivery devices comprises a container holding region. The container holding region may be a region configured to hold or receive the medicament container of the drug delivery device.

For example, the drug delivery device comprises at least one container holder element configured to abut against the medicament container or a medicament container holder holding the medicament container, e.g. in proximal or distal direction. For example, a container holder element may be configured to abut against a flange of the medicament container. The container holder element(s) may be configured to prevent the medicament container and/or the medicament container holder from axial and/or rotational movement relative to the container holding region or relative to the housing, respectively. The container holder elements may be the same or substantially the same in all drug delivery devices. The container holder element(s) may be part of the housing, e.g. integrally formed with the housing. For example, the dimensions of the container holder element(s) and/or the positions of the container holder element(s) with respect to the housing may be the same or substantially the same in all drug delivery devices.

The container holding region, particularly the container holder element (s), may be axially and/or rotationally fixed with respect to the housing. For example, the container holding region may be defined by the region between a most proximal and a most distal container holder element or may be the region of the housing from the most proximal container holder element to the distal end of the housing. If the drug delivery device comprises a medicament container holder, the container holding region may also be defined by the extensions of this medicament container holder.

According to at least one embodiment, at least some of the plunger rods of different drug delivery devices have different lengths. Accordingly, when the drug delivery devices comprise medicament containers, at least some of the medicament containers of the drug delivery devices may comprise medicaments occupying different fill volumes in the medicament containers.

The dimensions of the medicament containers of the different drug delivery devices and/or their positions in the housing may be the same or substantially the same in all drug delivery devices. Also the used materials may be the same or substantially the same.

According to at least one embodiment, a position of the operation feature relative to the container holding region is the same or substantially the same in each drug delivery device of the plurality of drug delivery devices. The position of the container holding region may be the position of the center or the distal end or the proximal end of the container holding region.

Hereinafter, the drug delivery device described herein, the plunger rod described herein, the set of plunger rods described herein, the set of drug delivery devices described herein and the method for assembling a drug delivery device described herein will be explained in more detail with reference to drawings on the basis of exemplary embodiments. Same reference signs indicate same elements in the individual figures. However, the size ratios involved are not necessarily to scale, individual elements may rather be illustrated with exaggerated size for a better understanding.

### BRIEF DESCRIPTION OF THE DRAWINGS

FIGS. 1 to 10 show a first exemplary embodiment of a drug delivery device in different views and different positions during usage of the drug delivery device,

FIGS. 11 to 14 show a second exemplary embodiment of the drug delivery device in different views and different positions during usage of the drug delivery device,

FIGS. 15 to 18 show a third exemplary embodiment of the drug delivery device in different views and different positions during usage of the drug delivery device,

FIGS. 19 and 20 show an exemplary embodiment of an indicator in different states,

FIG. 21 shows a section of an exemplary embodiment of a plunger rod,

FIG. 22 shows a section of an exemplary embodiment of a release member,

FIG. 23 shows an exemplary embodiment of a method for assembling a drug delivery device as well as an exemplary embodiment of a set of plunger rods and an exemplary embodiment of a set of drug delivery devices,

FIG. 24 shows a further exemplary embodiment of a set of plunger rods.

### DESCRIPTION OF EXEMPLARY EMBODIMENTS

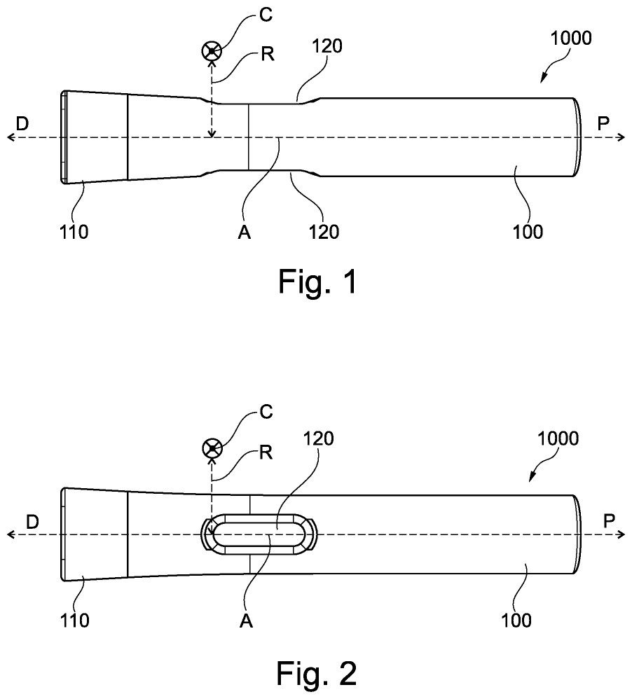

FIGS. 1 and 2 show side views of a first exemplary embodiment of the drug delivery device 1000. FIG. 1 shows a first view of the drug delivery device 1000 and FIG. 2 shows a second view in which the device 1000 is rotated by 90° around a longitudinal axis A compared to the first view.

FIGS. 1 and 2 also indicate the coordinate system used herein for specifying positions of members or elements or features. The distal direction D and proximal direction P run parallel to the longitudinal axis A. The longitudinal axis A is a main extension axis of the device 1000. The radial direction R is a direction perpendicular to the longitudinal axis A and intersecting with the longitudinal axis A. The azimuthal direction C, also referred to as angular direction or rotational direction, is a direction perpendicular to the radial direction R and to the longitudinal axis A. The different directions and axes will not be indicated in every of the following figures in order to increase the clarity of the figures.

The drug delivery device 1000 according to the first exemplary embodiment is an auto-injector. The auto-injector 1000 comprises a housing 100. A cap 110 is removably attached or coupled to the housing 100 at a distal end of the housing 100. The housing 100 may be formed in one piece and may extend from the cap 110 to the proximal end of the auto-injector 1000. The housing 100 is a cylindrically-shaped sleeve.

As can be further seen in FIGS. 1 and 2, the housing 100 comprises windows 120 through which a medicament container inside the housing 100 can be investigated. For example, the fill level of the drug inside the medicament container or the advancement of a stopper in the medicament container or the drug clarity or the degradation of the drug can be observed through the windows 120.

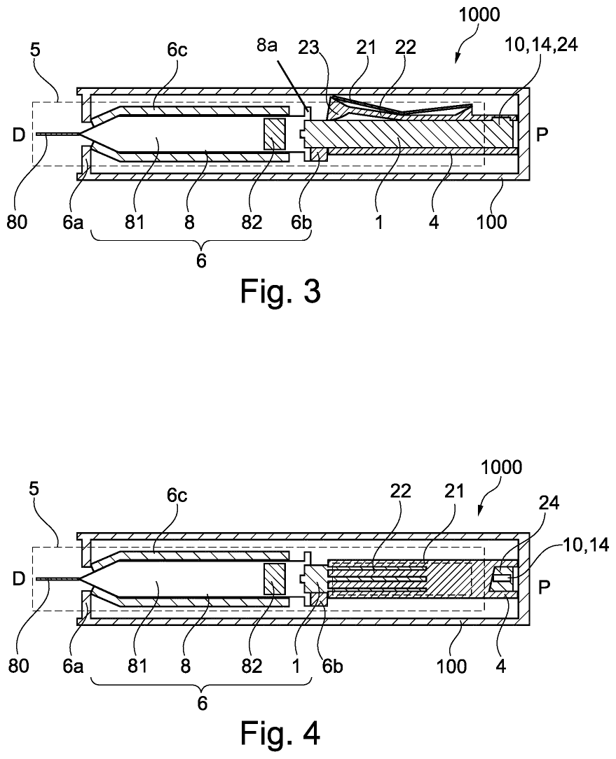

FIGS. 3 and 4 show the auto-injector 1000 in the same views as in FIGS. 1 and 2, but now in cross-sectional views and the cap 110 removed from the housing 100. A medicament container 8 in the form of a syringe 8 is received in the housing 100, namely in a container holding region 6. In an embodiment, a flange 8a is located in a proximal portion of the syringe 8a, e.g. at the proximal end of the syringe 8. The flange 8a may protrude radially from the syringe 8, e.g. from a barrel of the syringe. The syringe 8 is held in a medicament container holder 6c, also referred to as syringe holder 6c. Container holder elements 6a, 6b are provided which are axially and rotationally fixed to the housing 100. The container holder elements 6a, 6b abut against the flange 8a of the syringe 8 and/or a distal end of the syringe holder 6c. In this way the syringe 8 is axially and preferably also rotationally supported relative to or fixed to the housing 100. Consequently, the syringe 8 is not moved distally relative to the housing 100 for needle insertion and/or for a delivery operation. The container holder element 6a is formed integrally with the housing 100. In other embodiments, a separate container holder can be provided.

The syringe 8 comprises a cartridge 81 (or barrel) filled with a drug. The syringe 8 is sealed in proximal direction P by a stopper 82. The syringe 8 further comprises a needle 80 at the distal end of the medicament container 8. The needle 80 is fluidically coupled to the cartridge 81. A movement of the stopper 82 in distal direction D will press the drug in the cartridge 81 out of the needle 80. The cartridge may be formed of glass. The stopper 82 may be formed of a rubber and/or in one piece.

A plunger rod 1 is received in the housing 100. The plunger rod 1 is arranged proximally offset with respect to the stopper 82. A distal end of the plunger rod 1 projects into the syringe 8, particularly into the cartridge 81 and faces the stopper 82.

FIGS. 3 and 4 show the auto-injector 1000 in an initial state. In the initial state, the plunger rod 1 is spaced from the stopper 82, particularly in order to take into account manufacturing tolerances, assembling tolerances and/or movement of the stopper 82 during transport. In the initial state, the distal end of the plunger rod 1 may be arranged within the syringe 8 (see FIG. 3) or outside of the syringe 8 (not explicitly shown). In the initial state, the distal end of the plunger rod 1 may be proximally offset from the proximal end of the stopper 82. Preferably, the auto-injector 100 and/or the plunger rod 1 is designed to ensure that there is a distance between the plunger rod 1, preferably a distal end thereof, and the stopper 82, preferably a proximal end thereof in the initial state in all tolerance conditions.

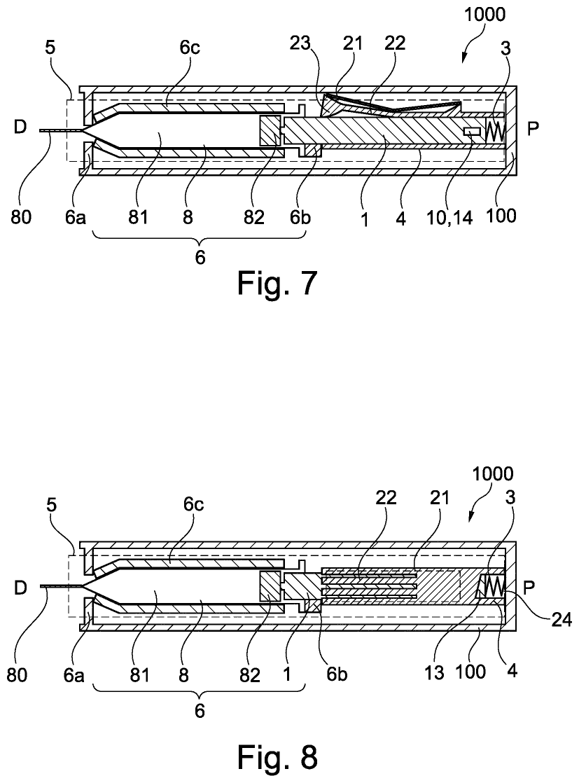

The plunger rod 1 is biased in distal direction D by a drive spring 3 (see, e.g., FIGS. 7 to 10). The drive spring 3 is a compression spring which is compressed in the initial state and presses against the plunger rod 1 trying to move the plunger rod 1 in distal direction D. However, in the initial state, a distal movement of the plunger rod 1 is prevented due to a coupling of the plunger rod 1 to the housing 100 by an axial-lock interface.

The axial-lock interface is established between the plunger rod 1 and a drive spring holder 4. The drive spring holder 4 is axially, preferably also rotationally, fixed with respect to the housing 100. The plunger rod 1 has a holding structure 14, the holding structure 14 comprising a protrusion 10. The protrusion 10 projects from a main body of the plunger rod 1 in radial outward direction. In the initial state, the protrusion 10 projects or engages into a recess 24 of the drive spring holder 4, respectively, and abuts against an edge delimiting the recess 24 in distal direction D. In this way, the axial-lock interface is established and the plunger rod 1 is prevented from a movement in distal direction D induced by the drive spring 3.

As can be further be seen in FIGS. 3 and 4, the drive spring holder 4 comprises a support element 22 in the form of a resilient arm 22. The resilient arm 22 is orientated axially and is displaceable in radial direction. A proximal end of the resilient arm 22 is fixed to a main body of the drive spring holder 4 and a distal end of the resilient arm 22 is displaceable in radial direction. An operation feature 23 in form of a protrusion 23 is located at the distal end of the resilient arm 22 and protrudes from the resilient arm 22 in radial inward direction. The protrusion 23 abuts against an outer surface, namely a lateral surface, of the plunger rod 1. In FIGS. 3 and 4 the resilient arm 22 and the protrusion 23 are in a first radial position and are biased in radial inward direction. The resilient arm 22 with the protrusion 23 is held in the first radial position due to the abutment against the plunger rod 1.

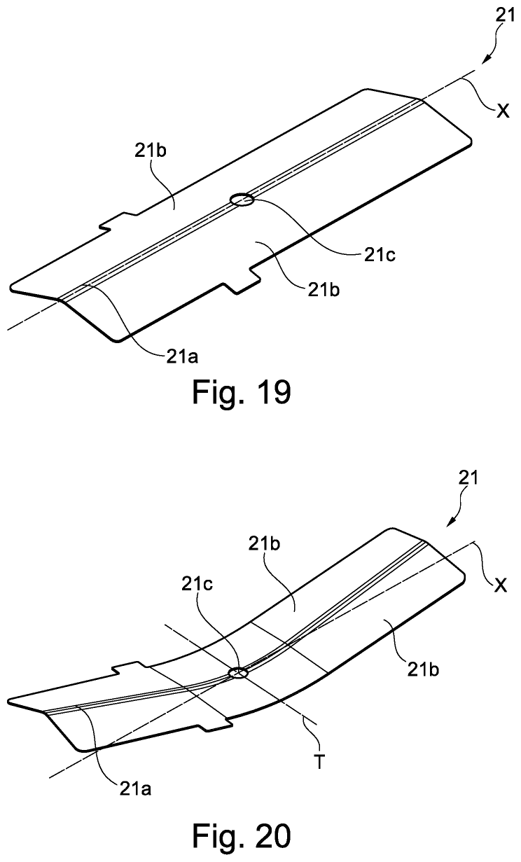

The resilient arm 22 supports and holds an indicator 21 in form of a leaf spring 21 in a first state or biased state, respectively. FIG. 20 shows the leaf spring 21 in the biased state in detail. The indicator 21 is configured to switch from the first state into a second state, which may be a relaxed state. The indicator 21 in the relaxed state is shown in FIG. 19. When switching from the first state into the second state, the indicator 21 creates an audible and/or tactile feedback due to energy being released. In the initial state, shown in FIGS. 3 and 4, however, the indicator 21 is prevented from switching from the first state into the second state due to the support element 22 held in the first position and thereby holding the indicator 21 in the first state. The indicator 21 and the resilient arm 22 with the protrusion 23 are part of an audible and/or tactile feedback mechanism which is operatively coupled to the plunger rod 1.

As can be further seen in FIGS. 3 and 4, the auto-injector 1000 comprises a release member 5 in form of a needle shroud 5. The needle shroud 5 is received in the housing 100 and telescopically coupled thereto. In the initial state, the needle shroud 5 is in an extended position in which it distally projects out of the housing 100 and beyond the needle 80 thereby covering the needle 80.

The needle shroud 5 in the extended position is coupled to the plunger rod 1 via a rotation-lock interface which prevents a rotation of the plunger rod 1 with respect to the release member 5 or with respect to the housing 100, respectively. A rotation of the plunger rod 5 would release the axial-lock interface as will be explained below. Indeed, the plunger rod 1 may be biased in rotational direction, e.g. by the drive spring 3 pressing the plunger rod 1 in distal direction D against an inclined surface of the drive spring holder 4 which induces a torque onto the plunger rod 1. In this exemplary embodiment the inclined surface delimits the recess 24 in distal direction and the protrusion 10 is pressed against this inclined surface.

In FIG. 4 one can see that the drive spring holder 4 actually comprises two separate support elements 22 each in the form of a resilient arm 22.

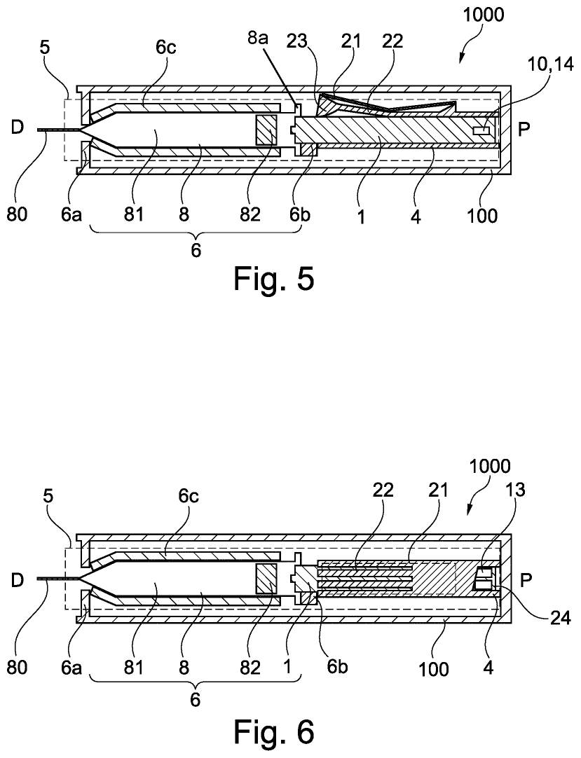

FIGS. 5 and 6 show the auto-injector 1000 in the same views as in FIGS. 3 and 4 and in a position during usage of the auto-injector 1000. The distal end of the auto-injector 1000 is, e.g., pressed against an injection side of a skin region of a body. This causes the needle shroud 5 to move in proximal direction P with respect to the housing 100 into a retracted position and thereby exposing the needle 80. The needle 80 is, e.g., pierced into a tissue of the body. The movement of the needle shroud 5 in the retracted position releases the rotation-lock interface. The plunger rod 1 rotates automatically as it is biased in rotational direction. This, in turn, releases the axial-lock interface since the protrusion 10 disengages from the recess 24. A movement of the plunger rod 1 in distal direction D is now no longer prevented. The auto-injector 1000 is now in a released state.

As can be seen in FIG. 6, the plunger rod 1 comprises two trigger features 13 each in the form of a recess or cut-out in the plunger rod 1. Due to the rotation of the plunger rod 1, the recesses 13 are now aligned with the protrusions 23 in rotational direction.

FIGS. 7 and 8 show the auto-injector 1000 in the same views as in FIGS. 5 and 6 but at a later position during usage of the auto-injector 1000. The plunger rod 1 has moved in distal direction D due to the energy provided by the drive spring 3. In FIGS. 7 and 8, the plunger rod 1 is in an interaction position in which it hits against the stopper 82. When hitting the stopper 82, an impulse is transferred to the syringe 8. However, the initial distance between the plunger rod 1 and the stopper 82 in the initial state is chosen such that a probability of a damage of the syringe 8, particularly of the cartridge 81, is reduced. For example, in at most 1 out of 10,000 of such drug delivery devices a damage of the syringe 8 occurs.

The plunger rod 1 hitting the stopper 82 pushes the stopper 82 in distal direction D. The syringe 8 or the needle 80, respectively, is not moved in distal direction as it is held in position by the container holder elements 6a, 6b and the syringe holder 6c.

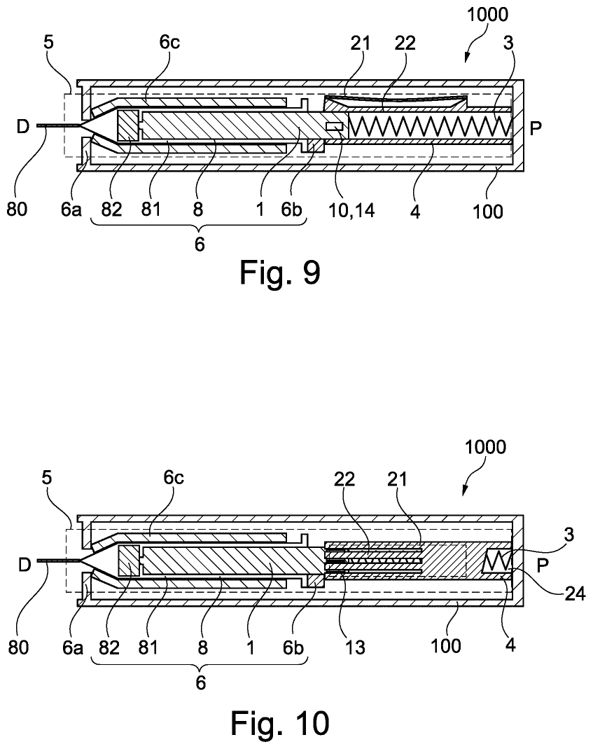

FIGS. 9 and 10 show the auto-injector 1000 in the same views as in FIGS. 7 and 8 but at an even later position during usage. The plunger rod 1, driven by the drive spring 3, has further moved in distal direction D and has thereby pushed the stopper 82 in distal direction D as well. Thereby, the drug stored in the cartridge 81 has been pressed out of the syringe 8 through the needle 80 and into the tissue of the body. The plunger rod 1 has reached an end position and cannot be moved further in distal direction D. This end position is, in this exemplary embodiment, also a feedback position in which the protrusions 23 and the recesses 13 rotationally and axially overlap with each other. This triggers or activates the feedback mechanism. In particular, this overlap allows the protrusions 23 to engage into the recesses 13 so that the resilient arms 22 with the protrusions 23 can displace from their respective first positions into second positions. The resilient arms 22 in the second positions do, however, no longer hold the indicator 21 in the first state. As a consequence of this, the indicator 21 is enabled to switch into the second state, which preferably happens automatically. Thereby, an audible and/or tactile feedback is created. This indicates the user the end of the delivery of the drug.

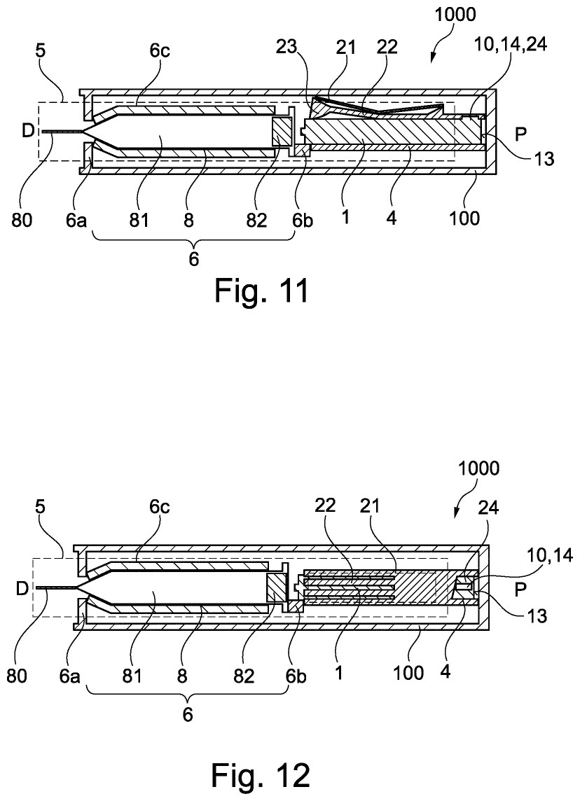

FIGS. 11 and 12 show a second exemplary embodiment of a drug delivery device 1000. The views are the same as in FIGS. 2 and 3. The drug delivery device is an auto-injector 1000. Moreover, also in FIGS. 11 and 12, the auto-injector 1000 is in the initial state. The auto-injector 1000 of the first and second exemplary embodiments are almost the same. A difference is that, in the second exemplary embodiment, the syringe 8 is filled with a greater volume of the drug. The geometry and the dimensions of the syringe 8 are, however, the same. Also the geometry, the dimensions and the relative positions of and between the housing 100, the container holding region 6, the container holder elements 6a, 6b, the syringe holder 6c, the syringe 8, the drive spring holder 4, the indicator 21, the resilient arms 22 and the protrusions 23 are the same or substantially the same in both exemplary embodiments. Particularly, only the plunger rod 1, the amount of drug in the syringe 8 and the start position of the stopper 82 are different in the two exemplary embodiments.

Due to the larger amount of the drug stored in the syringe 8, the stopper 82 is located further proximal in the second exemplary embodiment than in the first exemplary embodiment. Accordingly, the length of the plunger rod 1 is chosen smaller in the second exemplary embodiment than in the first exemplary embodiment in order to maintain an initial distance between the plunger rod 1 and the stopper 82 in the initial state.

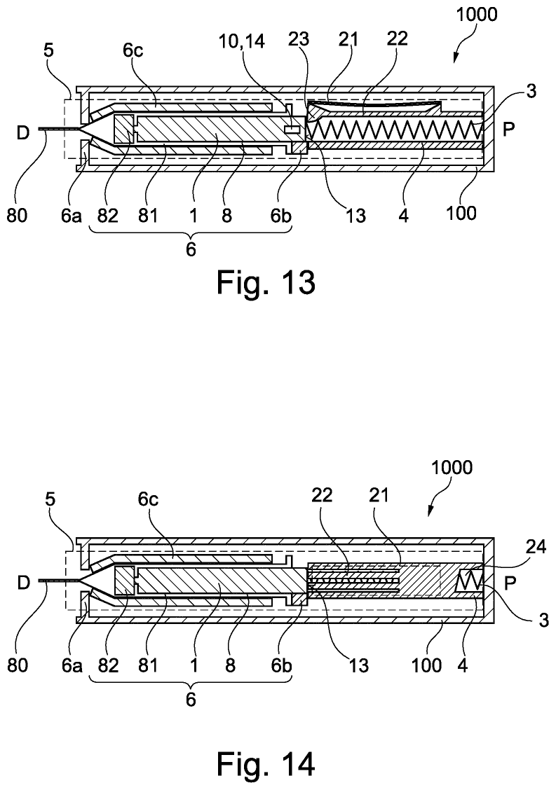

FIGS. 13 and 14 show the auto-injector 1000 in the same views as in FIGS. 11 and 12 but now after the auto-injector 1000 has been switched from the initial state into the released state and after the plunger rod 1 has been moved in distal direction D into the end position by the drive spring 3. In the second exemplary embodiment, the trigger feature 13 of the plunger rod 1 is formed by the proximal end of the plunger rod 1. As soon as the proximal end 13 of the plunger rod 1 passes the protrusions 23 of the resilient arms 22, the resilient arms 22 are enabled to displace radially inwardly whereby the protrusions 23 displace into the space proximally behind the plunger rod 1. The indicator 21 switches from its first state into the second state and creates the audible and/or tactile feedback.

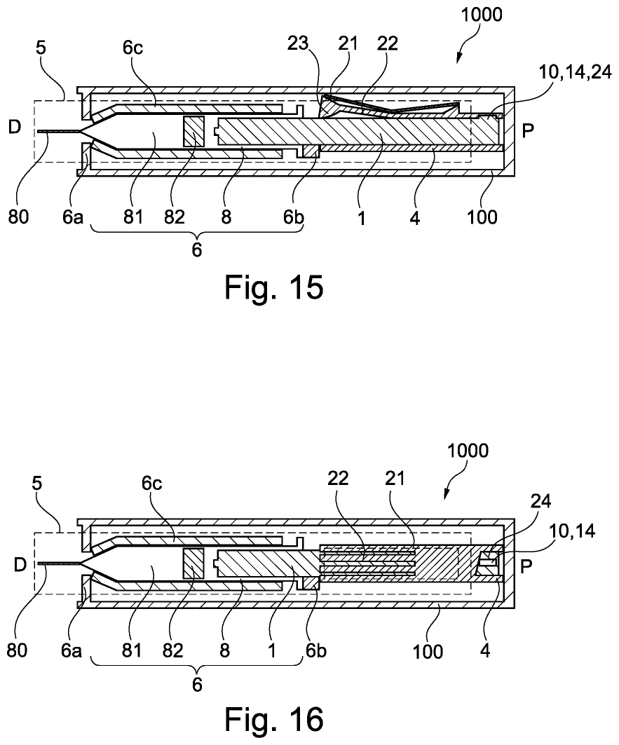

FIGS. 15 and 16 show a third exemplary embodiment of the drug delivery device 1000, again in the same views as in FIGS. 3 and 4. The drug delivery device 1000 of the third exemplary embodiment is again an auto-injector 1000. Again, the dimensions of and relative positions between the housing 1000, the syringe 8, the container holding region 6, the container holder elements 6a, 6b, the syringe holder 6c, the syringe 8, the drive spring holder 4, the indicator 21, the resilient arms 22 and the protrusions 23 are the same or substantially the same as in the first and second exemplary embodiment. The difference is again the fill volume of the drug stored in the syringe 8 and the design of the plunger rod 1. In the third exemplary embodiment, the fill volume is smaller than in the first exemplary embodiment. Accordingly, the stopper 82 is located further distal than in the first exemplary embodiment. As a consequence of this, the plunger rod 1 is chosen longer than in the first exemplary embodiment. Again, the length of the plunger rod 1 is chosen such that, in the initial state, the plunger rod 1 is axially spaced from the stopper 82 but close enough to the stopper 82 to reduce the risk of damage of the syringe 8 or the cartridge 81, respectively, when the plunger rod 1 hits the stopper 82 in the released state.

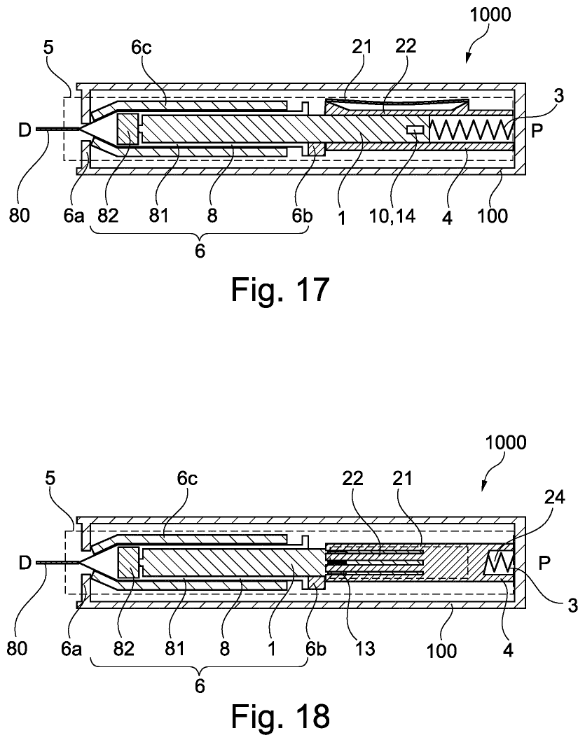

FIGS. 17 and 18 show the auto-injector 1000 of FIGS. 15 and 16 after the auto-injector 1000 has been switched from the initial state into the released state and after the plunger rod 1, driven by the drive spring 3, has been moved into the end position which may also the feedback position. Also in this case, the plunger rod 1 comprises recesses 13 forming trigger features 13. In the feedback position, the recesses 13 axially and rotationally overlap with the protrusions 23 of the resilient arms 22, allowing the protrusions 23 to engage into the recesses 13 so that the resilient arms 22 can displace and so that the indicator 21 can switch from its first state into the second state thereby creating the audible and/or tactile feedback.

A difference between the plunger rods 1 of the first exemplary embodiment and the third exemplary embodiment is that, in the third exemplary embodiment, the recesses 13 do not overlap with the protrusion 10 in axial direction. Instead, in the third exemplary embodiment, the recesses 13 are offset in distal direction D with respect to the protrusion 10.

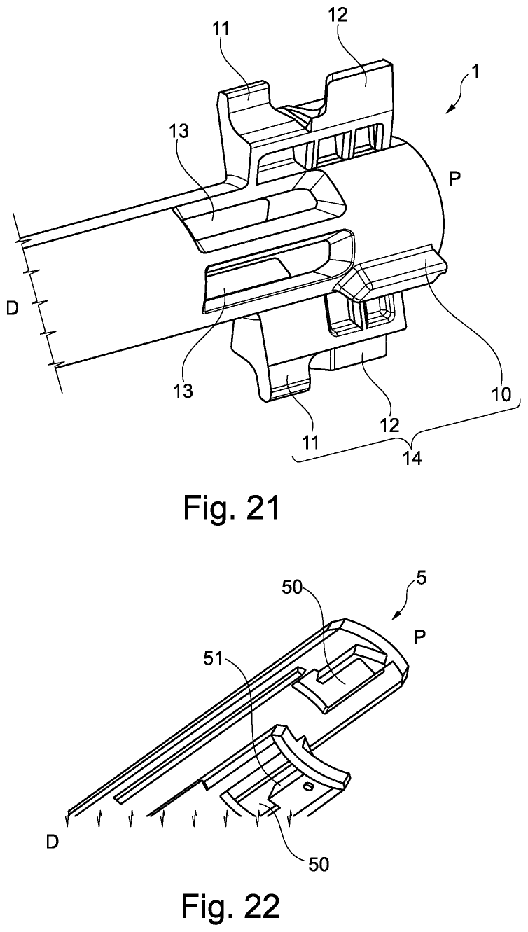

FIGS. 19 and 20 show an exemplary embodiment of the indicator 21 in a first state or biased state (FIG. 20) and in a second state or relaxed state (FIG. 21). The indicator 21 is a leaf spring 21, e.g. formed of a metal sheet. The leaf spring 21 is bent by a certain angle about a longitudinal axis X forming a longitudinal round fold 21a. Two angled wing-shaped sections 21b are arranged on both sides of the longitudinal round fold 21a. A notch 21c is formed in the leaf spring 21, namely in the longitudinal round fold 21a. The notch 21c may be provided to support consistency of priming, i.e. switching into the first (biased) state. In the biased state, the leaf spring 21 is bent around a transversal axis T running perpendicularly to the longitudinal axis X.

FIGS. 21 and 22 show proximal sections of an exemplary embodiment of the plunger rod 1 and of the needle shroud 5. The plunger rod 1 and the needle shroud 5 may be those of the first exemplary embodiment of the drug delivery device 1000. The plunger rod 1 comprises elongated recesses 13 into which the protrusions 23 of the resilient arms 22 can engage when the plunger rod 1 is in the feedback position. The plunger rod 1 further comprises a holding structure 14. The holding structure 14 is configured to couple to the drive spring holder 4 and the needle shroud 5 in order to establish the axial-lock interface and the rotation-lock interface. The holding structure 14 comprises a plurality of protrusions 10, 11, 12 protruding radially outwardly. A first protrusion 10 is configured to engage into a recess 24 of the drive spring holder 4 in order to establish the axial-lock interface. A second protrusion 11 is configured to engage into a recess 50 of the needle shroud 5 (see FIG. 22) and to abut against an edge 51 delimiting the recess 50 in rotational direction in order to establish the rotation-lock interface. A third protrusion 12 may abut against a ramp of the needle shroud 5 when the needle shroud is moved in proximal direction P. This abutment may support or guide the rotation of the plunger rod 1. The protrusions 10, 11, 12 project radially outwardly and are axially and rotationally offset with respect to each other.

The protrusion 11, 12 and the recess 50 have not been illustrated in the previous figures in order to increase the clarity of the figures.

As can be seen in FIG. 22, the recess 50 comprises two sections, wherein a first section is delimited in rotational direction by the edge 51 and the second section, located further distal than the first section, has a larger extent in rotational direction. In the initial state, when the needle shroud 5 is in the extended position, the protrusion 11 projects into the first section and abuts against the edge 51. When the needle shroud 5 is moved in proximal direction P into its retracted position, the protrusion 11 axially overlaps with the second section of the recess 50. This allows a rotation of the plunger rod 1.

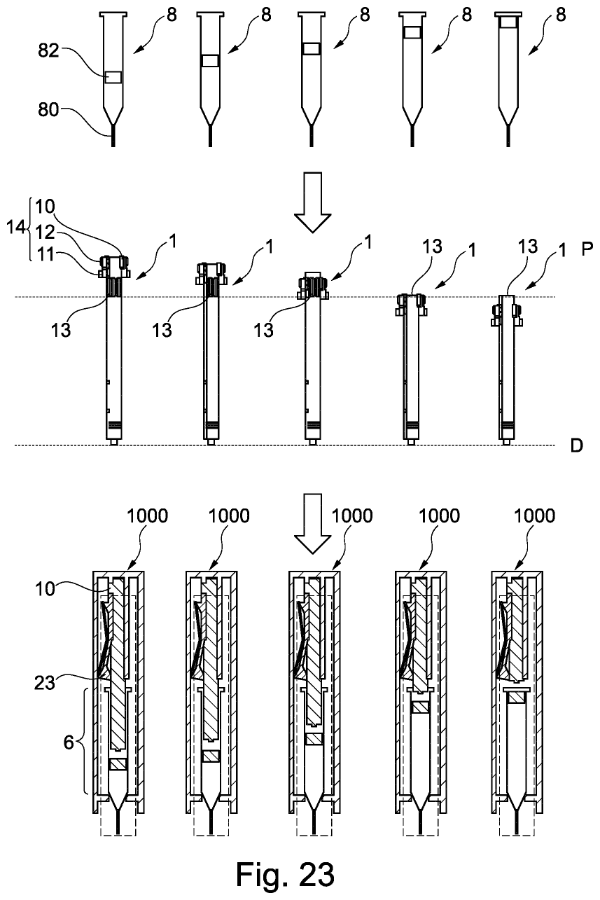

FIG. 23 illustrates an exemplary embodiment of a method for assembling a drug delivery device. A plurality of medicament containers 8 in the form of syringes 8 are provided. All syringes 8 have the same or substantially the same outer dimensions, particularly the same or diameters and the same substantially the same lengths. They may all be formed of the same material(s). They are, however, different with respect to the amount of medicament stored in the respective syringe 8. The more medicament is stored in the syringe 8, the further the stopper 82 is spaced from the needle 80.

Moreover, a set of plunger rods 1 is provided. The plunger rods 1 all have different lengths and all comprise a trigger feature 13 as well as a holding structure 14 with several protrusion 10, 11, 12. In some of the plunger rods 1, the trigger feature 13 is an elongated recess 13 and, in some plunger rods 1, the trigger feature 13 is a proximal end of the plunger rod 13. For all plunger rods 1, the distance between a distal end of the plunger rod 1 and the trigger feature(s) 13 is the same or substantially the same. In case the trigger feature 13 is a recess, the distance is, e.g., measured from the distal end of the recess 13 to the distal end of the plunger rod 1.

The holding structures 14 of the different plunger rods are the same or substantially the same for all plunger rods 1. Particularly, the relative positions between the protrusions 10, 11, 12 and the dimensions of the protrusions 10, 11, 12 are the same or substantially the same for all plunger rods 1. Depending on the length of the plunger rod 1, the holding structure 14 is offset in proximal direction P with respect to the trigger feature 13 or axially overlaps with the trigger feature 13 or is offset in distal direction D with respect to the trigger feature 13. In some plunger rods 1, the trigger feature 13 does not overlap with the holding structure 14 in axial direction.

In a first step of the method, a syringe 8 with a desired amount of the medicament is selected. Afterwards, a plunger rod 1 from the set of plunger rods is selected depending on the selected syringe 8. Then, the selected plunger rod 1 and the selected syringe 8 are used for assembling a drug delivery device 1000. In the lower part of FIG. 23, a set of drug delivery devices 1000 assembled in this way is shown. As can be seen, the dimensions of most of the components and the relative positions between the components are the same in all the drug delivery devices 1000. Particularly, the position of the protrusions 23 relative the container holding region 6 and/or relative to the container holder elements 6a, 6b is the same in all drug delivery devices 1000.

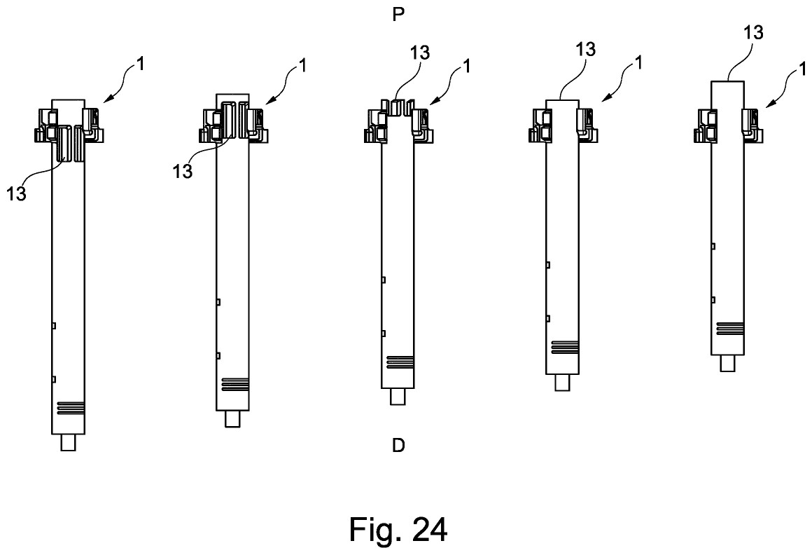

FIG. 24 shows a second exemplary embodiment of a set of the plunger rods. One plunger rod 1 (the one in the middle) comprises trigger features 13 in form of elongated recesses which are not delimited in proximal direction P by an edge of the plunger rod 1.

### Further Explanations and Definitions

The terms “drug” or “medicament” are used synonymously herein and describe a pharmaceutical formulation containing one or more active pharmaceutical ingredients or pharmaceutically acceptable salts or solvates thereof, and optionally a pharmaceutically acceptable carrier. An active pharmaceutical ingredient (“API”), in the broadest terms, is a chemical structure that has a biological effect on humans or animals. In pharmacology, a drug or medicament is used in the treatment, cure, prevention, or diagnosis of disease or used to otherwise enhance physical or mental well-being. A drug or medicament may be used for a limited duration, or on a regular basis for chronic disorders.

As described below, a drug or medicament can include at least one API, or combinations thereof, in various types of formulations, for the treatment of one or more diseases. Examples of API may include small molecules having a molecular weight of 500 Da or less; polypeptides, peptides and proteins (e.g., hormones, growth factors, antibodies, antibody fragments, and enzymes); carbohydrates and polysaccharides; and nucleic acids, double or single stranded DNA (including naked and cDNA), RNA, antisense nucleic acids such as antisense DNA and RNA, small interfering RNA (siRNA), ribozymes, genes, and oligonucleotides. Nucleic acids may be incorporated into molecular delivery systems such as vectors, plasmids, or liposomes. Mixtures of one or more drugs are also contemplated.

The drug or medicament may be contained in a primary package or “drug container” adapted for use with a drug delivery device. The drug container may be, e.g., a cartridge, syringe, reservoir, or other solid or flexible vessel configured to provide a suitable chamber for storage (e.g., short- or long-term storage) of one or more drugs. For example, in some instances, the chamber may be designed to store a drug for at least one day (e.g., 1 to at least 30 days). In some instances, the chamber may be designed to store a drug for about 1 month to about 2 years. Storage may occur at room temperature (e.g., about 20° C.), or refrigerated temperatures (e.g., from about −4° C. to about 4° C.). In some instances, the drug container may be or may include a dual-chamber cartridge configured to store two or more components of the pharmaceutical formulation to-be-administered (e.g., an API and a diluent, or two different drugs) separately, one in each chamber. In such instances, the two chambers of the dual-chamber cartridge may be configured to allow mixing between the two or more components prior to and/or during dispensing into the human or animal body. For example, the two chambers may be configured such that they are in fluid communication with each other (e.g., by way of a conduit between the two chambers) and allow mixing of the two components when desired by a user prior to dispensing. Alternatively or in addition, the two chambers may be configured to allow mixing as the components are being dispensed into the human or animal body.

The drugs or medicaments contained in the drug delivery devices as described herein can be used for the treatment and/or prophylaxis of many different types of medical disorders. Examples of disorders include, e.g., diabetes mellitus or complications associated with diabetes mellitus such as diabetic retinopathy, thromboembolism disorders such as deep vein or pulmonary thromboembolism. Further examples of disorders are acute coronary syndrome (ACS), angina, myocardial infarction, cancer, macular degeneration, inflammation, hay fever, atherosclerosis and/or rheumatoid arthritis. Examples of APIs and drugs are those as described in handbooks such as Rote Liste 2014, for example, without limitation, main groups 12 (anti-diabetic drugs) or 86 (oncology drugs), and Merck Index, 15th edition.

Examples of APIs for the treatment and/or prophylaxis of type 1 or type 2 diabetes mellitus or complications associated with type 1 or type 2 diabetes mellitus include an insulin, e.g., human insulin, or a human insulin analogue or derivative, a glucagon-like peptide (GLP-1), GLP-1 analogues or GLP-1 receptor agonists, or an analogue or derivative thereof, a dipeptidyl peptidase-4 (DPP4) inhibitor, or a pharmaceutically acceptable salt or solvate thereof, or any mixture thereof. As used herein, the terms “analogue” and “derivative” refers to a polypeptide which has a molecular structure which formally can be derived from the structure of a naturally occurring peptide, for example that of human insulin, by deleting and/or exchanging at least one amino acid residue occurring in the naturally occurring peptide and/or by adding at least one amino acid residue. The added and/or exchanged amino acid residue can either be codable amino acid residues or other naturally occurring residues or purely synthetic amino acid residues. Insulin analogues are also referred to as “insulin receptor ligands”. In particular, the term “derivative” refers to a polypeptide which has a molecular structure which formally can be derived from the structure of a naturally occurring peptide, for example that of human insulin, in which one or more organic substituent (e.g. a fatty acid) is bound to one or more of the amino acids. Optionally, one or more amino acids occurring in the naturally occurring peptide may have been deleted and/or replaced by other amino acids, including non-codeable amino acids, or amino acids, including non-codeable, have been added to the naturally occurring peptide. Examples of insulin analogues are Gly(A21), Arg(B31), Arg(B32) human insulin (insulin glargine); Lys(B3), Glu(B29) human insulin (insulin glulisine); Lys(B28), Pro(B29) human insulin (insulin lispro); Asp(B28) human insulin (insulin aspart); human insulin, wherein proline in position B28 is replaced by Asp, Lys, Leu, Val or Ala and wherein in position B29 Lys may be replaced by Pro; Ala(B26) human insulin; Des(B28-B30) human insulin; Des(B27) human insulin and Des(B30) human insulin.

Examples of insulin derivatives are, for example, B29-N-myristoyl-des(B30) human insulin, Lys(B29) (N-tetradecanoyl)-des(B30) human insulin (insulin detemir, Levemir®); B29-N-palmitoyl-des(B30) human insulin; B29-N-myristoyl human insulin; B29-N-palmitoyl human insulin; B28-N-myristoyl LysB28ProB29 human insulin; B28-N-palmitoyl-LysB28ProB29 human insulin; B30-N-myristoyl-ThrB29LysB30 human insulin; B30-N-palmitoyl-ThrB29LysB30 human insulin; B29-N—(N-palmitoyl-gamma-glutamyl)-des(B30) human insulin, B29-N-omega-carboxypentadecanoyl-gamma-L-glutamyl-des(B30) human insulin (insulin degludec, Tresiba®); B29-N—(N-lithocholyl-gamma-glutamyl)-des(B30) human insulin; B29-N-(ω-carboxyheptadecanoyl)-des(B30) human insulin and B29-N-(ω-carboxyheptadecanoyl) human insulin.

Examples of GLP-1, GLP-1 analogues and GLP-1 receptor agonists are, for example, Lixisenatide (Lyxumia®), Exenatide (Exendin-4, Byetta®, Bydureon®, a 39 amino acid peptide which is produced by the salivary glands of the Gila monster), Liraglutide (Victoza®), Semaglutide, Taspoglutide, Albiglutide (Syncria®), Dulaglutide (Trulicity®), rExendin-4, CJC-1134-PC, PB-1023, TTP-054, Langlenatide/HM-11260C (Efpeglenatide), HM-15211, CM-3, GLP-1 Eligen, ORMD-0901, NN-9423, NN-9709, NN-9924, NN-9926, NN-9927, Nodexen, Viador-GLP-1, CVX-096, ZYOG-1, ZYD-1, GSK-2374697, DA-3091, MAR-701, MAR709, ZP-2929, ZP-3022, ZP-DI-70, TT-401 (Pegapamodtide), BHM-034. MOD-6030, CAM-2036, DA-15864, ARI-2651, ARI-2255, Tirzepatide (LY3298176), Bamadutide (SAR425899), Exenatide-XTEN and Glucagon-Xten.

An example of an oligonucleotide is, for example: mipomersen sodium (Kynamro®), a cholesterol-reducing antisense therapeutic for the treatment of familial hypercholesterolemia or RG012 for the treatment of Alport syndrome.

Examples of DPP4 inhibitors are Linagliptin, Vildagliptin, Sitagliptin, Denagliptin, Saxagliptin, Berberine.

Examples of hormones include hypophysis hormones or hypothalamus hormones or regulatory active peptides and their antagonists, such as Gonadotropine (Follitropin, Lutropin, Choriongonadotropin, Menotropin), Somatropine (Somatropin), Desmopressin, Terlipressin, Gonadorelin, Triptorelin, Leuprorelin, Buserelin, Nafarelin, and Goserelin.

Examples of polysaccharides include a glucosaminoglycane, a hyaluronic acid, a heparin, a low molecular weight heparin or an ultra-low molecular weight heparin or a derivative thereof, or a sulphated polysaccharide, e.g. a poly-sulphated form of the above-mentioned polysaccharides, and/or a pharmaceutically acceptable salt thereof. An example of a pharmaceutically acceptable salt of a poly-sulphated low molecular weight heparin is enoxaparin sodium. An example of a hyaluronic acid derivative is Hylan G-F 20 (Synvisc®), a sodium hyaluronate.

The term “antibody”, as used herein, refers to an immunoglobulin molecule or an antigen-binding portion thereof. Examples of antigen-binding portions of immunoglobulin molecules include F(ab) and F(ab′)2 fragments, which retain the ability to bind antigen. The antibody can be polyclonal, monoclonal, recombinant, chimeric, de-immunized or humanized, fully human, non-human, (e.g., murine), or single chain antibody. In some embodiments, the antibody has effector function and can fix complement. In some embodiments, the antibody has reduced or no ability to bind an Fc receptor. For example, the antibody can be an isotype or subtype, an antibody fragment or mutant, which does not support binding to an Fc receptor, e.g., it has a mutagenized or deleted Fc receptor binding region. The term antibody also includes an antigen-binding molecule based on tetravalent bispecific tandem immunoglobulins (TBTI) and/or a dual variable region antibody-like binding protein having cross-over binding region orientation (CODV).

The terms “fragment” or “antibody fragment” refer to a polypeptide derived from an antibody polypeptide molecule (e.g., an antibody heavy and/or light chain polypeptide) that does not comprise a full-length antibody polypeptide, but that still comprises at least a portion of a full-length antibody polypeptide that is capable of binding to an antigen. Antibody fragments can comprise a cleaved portion of a full length antibody polypeptide, although the term is not limited to such cleaved fragments. Antibody fragments that are useful in the present invention include, for example, Fab fragments, F(ab′)2 fragments, scFv (single-chain Fv) fragments, linear antibodies, monospecific or multispecific antibody fragments such as bispecific, trispecific, tetraspecific and multispecific antibodies (e.g., diabodies, triabodies, tetrabodies), monovalent or multivalent antibody fragments such as bivalent, trivalent, tetravalent and multivalent antibodies, minibodies, chelating recombinant antibodies, tribodies or bibodies, intrabodies, nanobodies, small modular immunopharmaceuticals (SMIP), binding-domain immunoglobulin fusion proteins, camelized antibodies, and VHH containing antibodies. Additional examples of antigen-binding antibody fragments are known in the art.

The terms “Complementarity-determining region” or “CDR” refer to short polypeptide sequences within the variable region of both heavy and light chain polypeptides that are primarily responsible for mediating specific antigen recognition. The term “framework region” refers to amino acid sequences within the variable region of both heavy and light chain polypeptides that are not CDR sequences, and are primarily responsible for maintaining correct positioning of the CDR sequences to permit antigen binding. Although the framework regions themselves typically do not directly participate in antigen binding, as is known in the art, certain residues within the framework regions of certain antibodies can directly participate in antigen binding or can affect the ability of one or more amino acids in CDRs to interact with antigen.

Examples of antibodies are anti PCSK-9 mAb (e.g., Alirocumab), anti IL-6 mAb (e.g., Sarilumab), and anti IL-4 mAb (e.g., Dupilumab).

Pharmaceutically acceptable salts of any API described herein are also contemplated for use in a drug or medicament in a drug delivery device. Pharmaceutically acceptable salts are for example acid addition salts and basic salts.

Those of skill in the art will understand that modifications (additions and/or removals) of various components of the APIs, formulations, apparatuses, methods, systems and embodiments described herein may be made without departing from the full scope and spirit of the present invention, which encompass such modifications and any and all equivalents thereof.

An example drug delivery device may involve a needle-based injection system as described in Table 1 of section 5.2 of ISO 11608-1:2014(E). As described in ISO 11608-1:2014(E), needle-based injection systems may be broadly distinguished into multi-dose container systems and single-dose (with partial or full evacuation) container systems. The container may be a replaceable container or an integrated non-replaceable container.

As further described in ISO 11608-1:2014(E), a multi-dose container system may involve a needle-based injection device with a replaceable container. In such a system, each container holds multiple doses, the size of which may be fixed or variable (pre-set by the user). Another multi-dose container system may involve a needle-based injection device with an integrated non-replaceable container. In such a system, each container holds multiple doses, the size of which may be fixed or variable (pre-set by the user).

As further described in ISO 11608-1:2014(E), a single-dose container system may involve a needle-based injection device with a replaceable container. In one example for such a system, each container holds a single dose, whereby the entire deliverable volume is expelled (full evacuation). In a further example, each container holds a single dose, whereby a portion of the deliverable volume is expelled (partial evacuation). As also described in ISO 11608-1:2014(E), a single-dose container system may involve a needle-based injection device with an integrated non-replaceable container. In one example for such a system, each container holds a single dose, whereby the entire deliverable volume is expelled (full evacuation). In a further example, each container holds a single dose, whereby a portion of the deliverable volume is expelled (partial evacuation).

The invention described herein is not limited by the description in conjunction with the exemplary embodiments. Rather, the invention comprises any new feature as well as any combination of features, particularly including any combination of features in the patent claims, even if said feature or said combination per se is not explicitly stated in the patent claims or exemplary embodiments.

### REFERENCE NUMERALS

1 plunger rod
3 drive spring
4 drive spring holder
5 needle shroud
6 container holding region
6a container holder element
6b container holder element
6c medicament container holder/syringe holder
8 medicament container/syringe
8a flange
10 protrusion
11 protrusion
12 protrusion
13 trigger feature
14 holding structure
21 indicator/leaf spring
21a longitudinal round fold
21b wing-shaped section
21c notch
22 support element
23 operation feature
24 recess
80 needle
81 cartridge
82 stopper
100 housing
110 cap
120 window
1000 drug delivery device
D distal direction
P proximal direction
A longitudinal axis
R radial direction
C azimuthal direction/rotational direction/angular direction
X longitudinal axis
T transversal axis

1. Drug delivery device (1000), comprising
a housing (100) configured to receive a medicament container (8) with a stopper (82) proximally sealing the medicament container (8),
a plunger rod (1) arranged axially moveable with respect to the housing (100) and/or the medicament container (8),
a feedback mechanism operatively coupled to the plunger rod (1), wherein
the drug delivery device (1000) has an initial state in which the plunger rod (1) is in a start position,
the drug delivery device (1000) is configured such that
in the initial state and when the medicament container (8) is received in the housing (100), the plunger rod (1) is axially spaced from the stopper (82),
the plunger rod (1) is axially movable from its start position in a distal direction (D) into a feedback position,
the movement of the plunger rod (1) in the distal direction (D) enables the plunger rod (1) to interact with the stopper (82) in order to push the stopper (82) in the distal direction (D) such that a medicament stored in the medicament container (8) is delivered,
the feedback mechanism is triggered when the plunger rod (1) reaches the feedback position,
triggering of the feedback mechanism creates an audible and/or tactile feedback in order to indicate an end of the medicament delivery.

2. Drug delivery device (1000) according to claim 1, wherein the drug delivery device (1000) is configured such that, when the medicament container (8) is received in the housing (100)
the plunger rod (1) is moveable from its start position in the distal direction (D) by a travel distance before interacting with the stopper (82) and, after having been moved by the travel distance and before reaching the feedback position, the plunger rod (1) interacts with the stopper (82),
in the initial state, an initial distance between the plunger rod (1) and the stopper (82) is chosen such that the probability of a damage of the medicament container (8) upon interaction of the plunger rod (1) with the stopper (8) is reduced.

3. Drug delivery device (1000) according to claim 1 or 2, wherein
in the initial state, an initial distance between the plunger rod (1) and the stopper (82) is chosen such that variations in the distance due to manufacturing tolerances and/or assembling tolerances and/or fluctuations occurring during a transport of the drug delivery device (1000) are taken into account.

4. Drug delivery device (1000) according to any one of the preceding claims, wherein
the feedback mechanism comprises an indicator (21) having a first state and a second state,
the drug delivery device (1000) is configured such that, when the feedback mechanism is triggered, the indicator (21) switches from the first state into the second state which creates the audible and/or tactile feedback.

5. Drug delivery device according to claim 4, wherein
the indicator (21) is a resilient element, particularly a leaf spring.

6. Drug delivery device (1000) according to any one of the preceding claims, wherein
the feedback mechanism comprises a trigger feature (13) axially fixed to the plunger rod (1),
the feedback mechanism comprises an operation feature (23) axially fixed to the housing (100),
at least one of the trigger feature (13) and the operation feature (23) is displaceable,
the drug delivery device (1000) is configured such that, when the plunger rod (1) is moved from its start position into the feedback position, the trigger feature (13) and the operation feature (23) axially pass each other or at least axially overlap which each other, which enables the displaceable feature to displace in order to trigger the feedback mechanism.

7. Drug delivery device (1000) according to claim 6, wherein
the operation feature (23) is a protrusion,
the trigger feature (13) is a recess in the plunger rod (1) or a proximal end of the plunger rod (1),
when the plunger rod (1) reaches the feedback position, the protrusion displaces into the recess (13) or displaces into a space proximally behind the proximal end of the plunger rod (1).

8. Drug delivery device (1000) according to claim 4 or any one of claims 5 to 7 in its dependency of claim 4, wherein
the feedback mechanism comprises a support element (22) providing a mechanical support for the indicator (21) in order to hold the indicator (21) in the first state before the plunger rod (1) reaches the feedback position,
the mechanical support holding the indicator (21) in the first state is released when the plunger rod (1) reaches the feedback position so that the indicator (21) is enabled to switch into the second state.

9. Drug delivery device (1000) according to any one of the preceding claims, wherein
the plunger rod (1) is arranged rotatably with respect to the housing (100),
in the initial state, the plunger rod (1) is coupled to the housing (100) by an axial-lock interface which prevents an axial movement of the plunger rod (1) in the distal direction (D),
the drug delivery device (1000) is configured such that a rotation of the plunger rod (1) relative to the housing (100) releases the axial-lock interface and enables the movement of the plunger rod (1) in the distal direction (D).

10. Drug delivery device (1000) according to any one of the preceding claims, further comprising
an energy member (3) configured to provide energy to induce an axial movement of the plunger rod (1) in the distal direction (D),
the drug delivery device (1000) is configured to be switched from the initial state into a released state in which the plunger rod (1) moves in the distal direction (D) due to the energy provided by the energy member (3).

11. Drug delivery (1000) device according to claim 9 or claim 10 in its dependency of claim 9, further comprising
a release member (5) arranged axially moveable with respect to the housing (100), wherein
when the drug delivery device (1000) is in the initial state, movement of the release member (5) in an axial direction enables a rotation of the plunger rod (1) in order to release the axial-lock interface.

12. Drug delivery device according to claim 11, wherein
in the initial state, the release member (5) is in an extended position in order to cover a drug delivery element (80) of the medicament container (8),
movement of the release member (5) in an axial direction into a retracted position enables the rotation of the plunger rod (1),
the retracted position is a position for exposing the drug delivery element (80).

13. Set of plunger rods for drug delivery devices, wherein
each plunger rod (1) of the set of plunger rods comprises a trigger feature (13) configured to enable a triggering of an audible and/or tactile feedback mechanism of a drug delivery device when the plunger rod (1) is assembled in the drug delivery device and when the trigger feature (13) axially passes or at least axially overlaps with an operation feature (23) of the feedback mechanism,
each plunger rod (1) has a distal end,
at least some plunger rods (1) of the set of plunger rods have different lengths,
a distance between the distal end and the trigger feature (13) is the same or substantially the same for all plunger rods (1).

14. Method for assembling a drug delivery device (1000), comprising the steps
providing a medicament container (8) comprising a medicament which occupies a fill volume in the medicament container (8),
choosing a plunger rod (1) from the set of plunger rods according to claim 13 depending on the fill volume,
assembling the drug delivery device (1000) by using the medicament container (8) and the chosen plunger rod (1).

15. Set of drug delivery devices (1000), wherein
each drug delivery device (1000) of the set of drug delivery devices comprises a plunger rod (1) of the set of plunger rods according to claim 13,
each drug delivery device (1000) comprises an audible and/or tactile feedback mechanism with an operation feature (23),
each drug delivery device (1000) comprises a container holding region (6) configured to receive a medicament container,
at least some of the drug delivery devices (1000) of the set of drug delivery devices comprise plunger rods (1) with different lengths,
a position of the operation feature (23) relative to the container holding region (6) is the same or substantially the same in each drug delivery device (1000).
